SEQUENCE LISTING (5 sequences)

SEQ ID NO: 1 - protein, 490 aa, synthetic construct
  REGION 1..490: note = English
MHHHHHHMVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILATQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDANTPVSGNLKVEFYNSNPSDTTNSINPQFKVTNTGSSAIDLSKLTLRYYYTVDGQKDQTFWCDHAAIIGSNGSYNGITSNVKGTFVKMSSSTNNADTYLEISFTGGTLEPGAHVQIQGRFAKNDWSNYTQSNDYSFKSRSQFVEWDQVTAYLNGVLVWGKEPGGSVV

SEQ ID NO: 2 - DNA, 1485 bp, synthetic construct
  misc_feature 4..1473: note = English
  source 1..1485: mol_type = other DNA
gtatacgtggtggtggtggtggtgtaccataagtgagatcttctaaagcaaccgctgaccgcagtctggcgcccgatgttagacctagttcacgacctcgtcccaccgcaatcgtcggacaaagttttggacccacactcgcaatggggctaggtcgcataacaagactcgccacttttgccggacttttagctataagtgcactagtaaggcatgctcccagactcgccgctggtctacccggtttagcttttctagaagttccaccaaatgggccacctgctagtggtgaaattccaataggacgtgatgccatgggaccactaactgccgcaatggggcttgtactagctaataaagccagcaggcatgcttccgtaacgccaaaaactaccattcttttagtggcactggccatgggacaccttgccgttgttttagtaactgctcgcagactaattgggcctaccgtcggacgacaaggcacactggtagttgccacaatggccgaccgcagacacgcttgcataagaccgctgggtctggttgtcgggcgcagcacgcgcatcgcaacgctcggtttcgtagtaacgcatatggtactcggacccacgcctcttgtcgcaccgcatgtcgttgttgtcgtagcgctaaggctggttgaaatggtagtcgcactggtggctttaagacggccaatcgtactggttctggtcgcacctgacgtggtacatatagacgccactatcgtggctcacgtcgttggacgacgacgtcatgccatcgaagacgtgggttgacttggcacgcgactggccgtagcgccaactcgtcctgtttttgtgggttcttcacaagcgcgtccaattcgtttagatattttggggcggctaattcctgaaaccaccgaagttgaaatcggtctaggacggcctaggctcgttcggctcgtttgcatcgaagtaactcctgcgcttgtggggccactcgccattggactttcaacttaaaatgttgtcgttgggctcgctatggtggttgtcgtagttgggcgttaagttccaatggttgtggccatcgtcgcgctaactggactcgtttgactgggacgcaatgataatgtggcacctgccggtcttcctagtttggaaaaccacgctagtgcgccgctagtaaccatcgttgccgtcgatattgccatagtggtcgttgcacttcccgtggaagcaattttactcgtcgtcgtggttgttgcgcctgtggatggacctctagtcgaaatggccaccgtgggaccttggcccacgcgtgcaagtctaagttccggcaaagcgctttttgctgacctcgttgatatgggtctcgttgctaatgtcgaagttctcggcatcggtcaaacacctcaccctagttcactggcgcatagacttaccacacgaccacaccccatttcttggcccaccatcgcaacacattactttcgaa

SEQ ID NO: 3 - DNA, 513 bp, synthetic construct
  misc_feature 1..513: note = English
  source 1..513: mol_type = other DNA
taccataagtgagatcttctaaagcaaccgctgaccgcagtctggcgcccgatgttagacctagttcacgacctcgtcccaccgcaatcgtcggacaaagttttggacccacactcgcaatggggctaggtcgcataacaagactcgccacttttgccggacttttagctataagtgcactagtaaggcatgctcccagactcgccgctggtctacccggtttagcttttctagaagttccaccaaatgggccacctgctagtggtgaaattccaataggacgtgatgccatgggaccactaactgccgcaatggggcttgtactagctaataaagccagcaggcatgcttccgtaacgccaaaaactaccattcttttagtggcactggccatgggacaccttgccgttgttttagtaactgctcgcagactaattgggcctaccgtcggacgacaaggcacactggtagttgccacaatggccgaccgcagacacgcttgcataagaccgc

SEQ ID NO: 4 - DNA, 435 bp, Severe acute respiratory syndrome coronavirus 2
  misc_feature 1..435: note = English
  source 1..435: mol_type = other DNA
tgggtctggttgtcgggcgcagcacgcgcatcgcaacgctcggtttcgtagtaacgcatatggtactcggacccacgcctcttgtcgcaccgcatgtcgttgttgtcgtagcgctaaggctggttgaaatggtagtcgcactggtggctttaagacggccaatcgtactggttctggtcgcacctgacgtggtacatatagacgccactatcgtggctcacgtcgttggacgacgacgtcatgccatcgaagacgtgggttgacttggcacgcgactggccgtagcgccaactcgtcctgtttttgtgggttcttcacaagcgcgtccaattcgtttagatattttggggcggctaattcctgaaaccaccgaagttgaaatcggtctaggacggcctaggctcgttcggctcgtttgcatcgaagtaactcctg

SEQ ID NO: 5 - DNA, 501 bp, Acetivibrio thermocellus
  misc_feature 1..501: note = English
  source 1..501: mol_type = other DNA
cgcttgtggggccactcgccattggactttcaacttaaaatgttgtcgttgggctcgctatggtggttgtcgtagttgggcgttaagttccaatggttgtggccatcgtcgcgctaactggactcgtttgactgggacgcaatgataatgtggcacctgccggtcttcctagtttggaaaaccacgctagtgcgccgctagtaaccatcgttgccgtcgatattgccatagtggtcgttgcacttcccgtggaagcaattttactcgtcgtcgtggttgttgcgcctgtggatggacctctagtcgaaatggccaccgtgggaccttggcccacgcgtgcaagtctaagttccggcaaagcgctttttgctgacctcgttgatatgggtctcgttgctaatgtcgaagttctcggcatcggtcaaacacctcaccctagttcactggcgcatagacttaccacacgaccacaccccatttcttggcccaccatcgcaacac

(19) United States
(12) Patent Application Publication
(10) Pub. No.: US 2025/0003966 A1
(43) Pub. Date: Jan. 2, 2025

(54) A RECOMBINANT CONSTRUCT FOR SCREENING DRUGS AGAINST SARS-COV-2 SPIKE PROTEIN

(71) Applicant: COUNCIL OF SCIENTIFIC AND INDUSTRIAL RESEARCH, New Delhi (IN)

(72) Inventors: Upasana RAY, Kolkata (IN); Prem Prakash TRIPATHI, Kolkata (IN); Feroza BEGUM, Kolkata (IN); Amit Kumar SRIVASTAVA, Kolkata (IN)

(21) Appl. No.: 18/704,638

(22) PCT Filed: Oct. 27, 2022
(86) PCT No.: PCT/IN2022/050949

§ 371 Date: Apr. 25, 2024

(30) Foreign Application Priority Data
Oct. 28, 2021 (IN) 202111049482

(51) Int. Cl. G01N 33/569; G01N 33/68

(52) U.S. Cl. CPC G01N 33/56983; G01N 33/6845; G01N 2333/165; G01N 2500/04

(57) ABSTRACT

Trypsin/trypsin-like proteases have been reported to be facilitating SARS-COV-2 entry into host cells. Spike has protease cleavage sites between the S1 and S2 domains. Cleavage property of proteases can be used to design drug screening assays meant for screening antiviral candidates against spike cleavage. In the claimed invention we have developed a proof-of-concept assay system for screening drugs against proteases which cleave spike between S1 and S2. We designed a fusion substrate protein containing a reporter protein, the protease cleavage site between S1 and S2 and a cellulose binding domain. The substrate protein can be immobilized on cellulose due to the presence of cellulose binding domain (CBD). When proteases cleave the substrate. CBD remains bound to cellulose and the reporter protein is dislodged. The released reporter can be used as read out of protease activity.

### TECHNICAL FIELD OF THE INVENTION

The present invention relates to development of a reporter based in vitro trypsin/trypsin-like protease-based SARS-COV-2 spike protein cleavage assay system. The assay system involves a recombinant construct for screening drugs against sars-cov-2 spike protein.

### BACKGROUND OF THE INVENTION

Trypsin like proteases have been known to play an important role in facilitating coronavirus infections (SARS-COV, SARS-COV2, MERS-COV etc) [1-4]. These enzymes cleave the spike protein to promote interaction with the host receptors for entry in the cell. Coronaviruses are known to possess specific cleavage sites called S1/S2 and S2/S2′ that are specifically recognized by airway proteases for further processing. Kam et al [2] and Weber et al [1] studied SARS-COV and MERS-COV respectively to identify potential protease cleavage sites in spike proteins and could identify the two sites (S1/S2 and S2/S2′) to be responsible for the proteolytic processing. SARS-COV2 spike is also known to possess these two sites along with an extra multibasic cleavage site near S1/S2 that makes it more susceptible for recognition by different proteases [3,4,5]. As per Hoffmann et al, in SARS-COV2 spike, the S1/S2 cleavage site is extended from 676 to 688th residue and S2′ site extended from 811th to 818th residues which is similar to the SARS-COV and MERS-COV spike cleavage sequence [1,2,4]. Airway serine proteases like trypsin is known to be important in mediating Influenza A virus infection by cleaving its envelope glycoprotein and has also been shown to recognize SARS-COV and SARS-COV2 spike for cleavage [2,5]. Other serine proteases like type II transmembrane serine protease TTSPs has been extensively studied to be important in processing of coronaviral spike protein and enhancing its infectivity [1-4]. Similarly, there are other proteases like furin and cathepsin which are also known to cleave SARS-COV2 spike protein in specific regions for its downstream processing [5], hence, airway proteases and trypsin like proteases play major role in enhancing viral infectivity for coronaviruses and hence can be a potential target for antiviral therapeutics in near future.

OBJECTIVES OF THE INVENTION

The main object of present invention is development of a reporter based in vitro trypsin/trypsin-like protease-based SARS-COV-2 spike protein cleavage assay system. The assay system involves a recombinant construct for screening drugs against sars-cov-2 spike protein.

Yet another object of the present invention is a substrate used in the claimed assay system for trypsin/trypsin-like protease cleavage activity

### SUMMARY OF THE INVENTION

Accordingly, the present invention provides a reporter based in vitro trypsin/trypsin-like protease-based SARS-COV-2 spike protein cleavage assay system. The assay system involves a recombinant construct for screening drugs against sars-cov-2 spike protein.

In an embodiment of the invention, the substrate used has been designed involving SARS-COV-2 spike protein's trypsin/trypsin-like protease cleavage region, a reporter protein and a cellulose binding domain. The substrate also carries hexa histidine tag at one end for the purpose of protein purification. Methionine was added at the N terminus of hexa histidine for protein expression. The DNA construct corresponding to the substrate has been entirely codon modified for expression in bacterial system.

In an embodiment of the invention the claimed substrate meant for use in the assay system gets cleaved by trypsin/trypsin-like protease/s. Cellulose matrix/slurry traps cellulose binding domain releasing the reporter protein free from the cleaved substrate which can then be detected using reporter assay.

In another embodiment of the invention the reporter used for designing the substrate is nanoluciferase. Alternatively, other luciferases as well as fluorescent reporters may be used. Nanoluc luciferase used in the construct is a newer bioluminescent protein with a better light emission capacity and enhanced physical and biochemical properties [6]. Its specific activity is greater than any other previously reported luciferase (for eg. Firefly or Renilla). Due to its high sensitivity, the Nanoluc luciferase can elicit bioluminescent signal even at a very low amount as compared to other luciferase. The designed construct is highly potent in manifesting the bioluminescence when gets cleaved by the specific enzyme. The designed assay system is based on the principle that more efficiently an enzyme cleaves the substrate, more bioluminescent signal should be produced and if any enzyme specific inhibitor is introduced then the signal should decrease in proportion to the amount of drug used.

### ABBREVIATIONS

SARS-COV-2 Severe acute respiratory syndrome coronavirus 2

MERS Middle East respiratory syndrome

CBD Cellulose binding domain

SDS-PAGE Sodium dodecyl sulfate polyacrylamide gel electrophoresis

TBST Tris-buffered saline Tween 20

TMPRSS2 Transmembrane serine protease 2

CM Camostat Mesylate

CB Cathepsin B

### BRIEF DESCRIPTION OF THE DRAWING

FIG. 1 Schematic representation showing principles of claimed assay system

FIG. 2 Schematic diagram to show the various cleavage patterns that might be obtained and visualized on an SDS-PAGE gel stained with protein dye/s when a protease cleaves the spike protein

FIG. 3 Schematic diagram of design of the substrate used in the assay system and vector map of cloned substrate

FIG. 4 Validation of substrate expressing plasmid using restriction digestion

FIG. 5 Purified substrate protein on SDS-PSGE gel stained with protein dye

FIG. 6 Validation of purified substrate by western blot analyses using anti SARS-COV-2 S!/S2 and anti-His antibodies

FIG. 7 Nano-luciferase assay based cleavage assay result using the claimed substrate and trypsin enzyme

FIG. 8 Gel based cleavage assay result using the claimed substrate and trypsin enzyme

FIG. 9 Substrate treated with different amounts of TMPRSS2

FIG. 10 Substrate digested with TMPRSS2 both in absence and presence of camostat mesylate (TMPRSS2 inhibitor) in two different amounts (Nano luciferase assay), CM=Camostat Mesylate

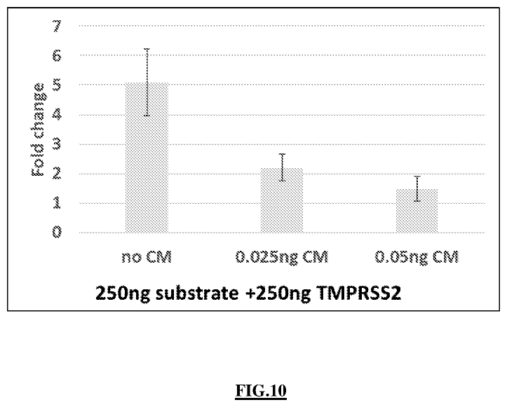

FIG. 11 Substrate treated with different units of Furin (50-800 mU) at 25 degC for 4 hours (SDS-PAGE gel). PL=protein ladder, S=substrate, F=Furin, BF-BSA digested with furin, F50-F80=different amounts of furin

FIG. 12 Substrate digested with Furin both in absence and presence of Furin inhibitor II (Furin inhibitor) in two different amounts (Nano luciferase assay)

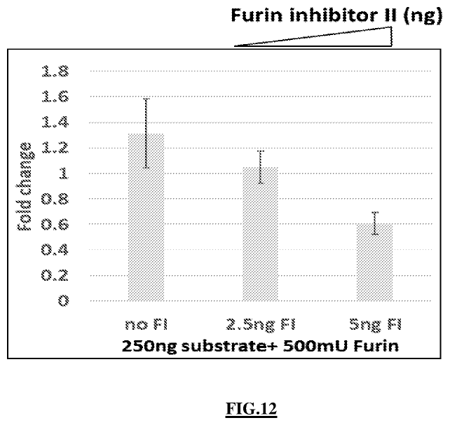

FIG. 13 Nanoluc luciferase assay result for the substrate digested with different amount of cathepsin B at 37 degC for 2 hrs. CB=Cathepsin B

FIG. 14 Effect of cathepsin B inhibitor on substrate digestion. CI=cathepsin B inhibitor

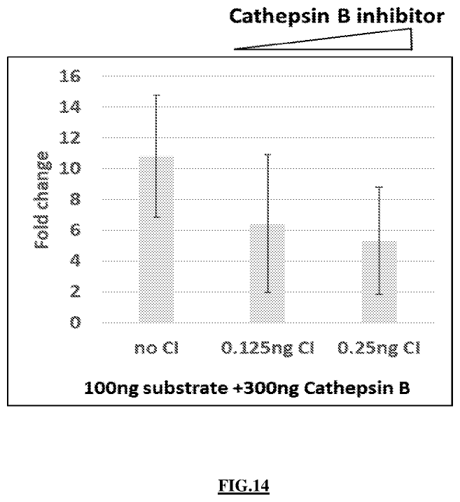

### DETAILED DESCRIPTION OF THE INVENTION

### Mode of Action of Claimed Drug Screening Assay System

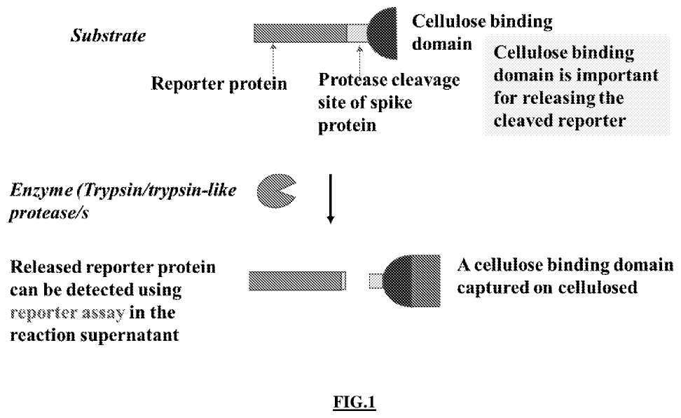

The substrate contains a reporter protein followed by spike protein cleavage sequence and a cellulose binding domain, all as fusion protein [FIG. 1]. Upon addition of the trypsin like protease, the substrate should get cleaved at the cleavage site and reporter protein dislodged.

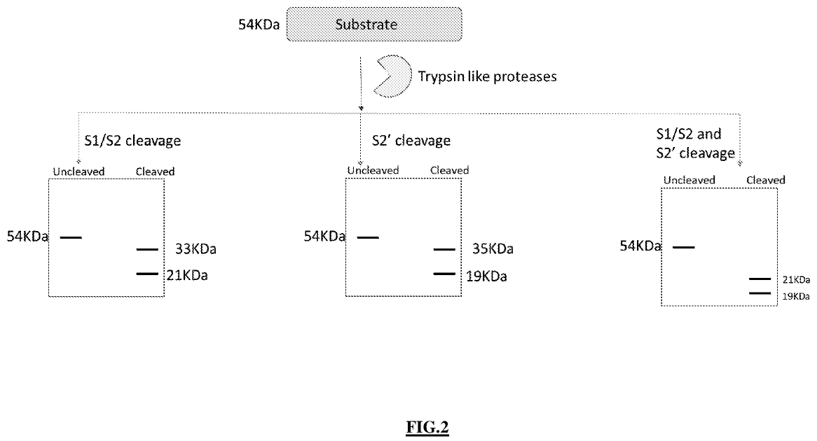

Cellulose from cellulose matrix/slurry when added to this reaction mix, will bind cellulose binding domain and the reporter protein will be available from the reaction supernatant for reporter assay. Positive reporter signal will indicate cleavage by protease. The cleavage bands may also be visualized on an SDS-PAGE after staining with protein staining dyes or western blot analysis [FIG. 2]

### Utility of the Assay System

This assay system can used to screen drugs against trypsin/trypsin-like protease/s which cleave SARS-COV-2 spike protein. Since such cleavage is important for the entry process of this virus in its host cell, the claimed drug screening assay system can be used to screen drugs against role of trypsin/trypsin-like protease/s in cleaving the spike protein. The drug is selected from the group comprising of Camostat mesylate, cathepsin B inhibitor, furin inhibitor II, protease inhibitors, Furin inhibitor I, trypsin inhibitors, Ovomucoid, Kunitz Trypsin Inhibitor, serine protease inhibitors, TMPRSS2 inhibitors. The designed construct may help in identifying cleavage sites of the proteases that are not yet known to cleave spike. It can also be used to identify if any other unknown host protease involved in spike protein cleavage. The designed assay system is easy to understand and interpret with no complicated steps involved.

### EXAMPLES

The following examples are given by way of illustration of the present invention and therefore should not be construed to limit the scope of the present invention

### Example 1

### Reporter Based Substrate DNA: Construct Design and Cloning

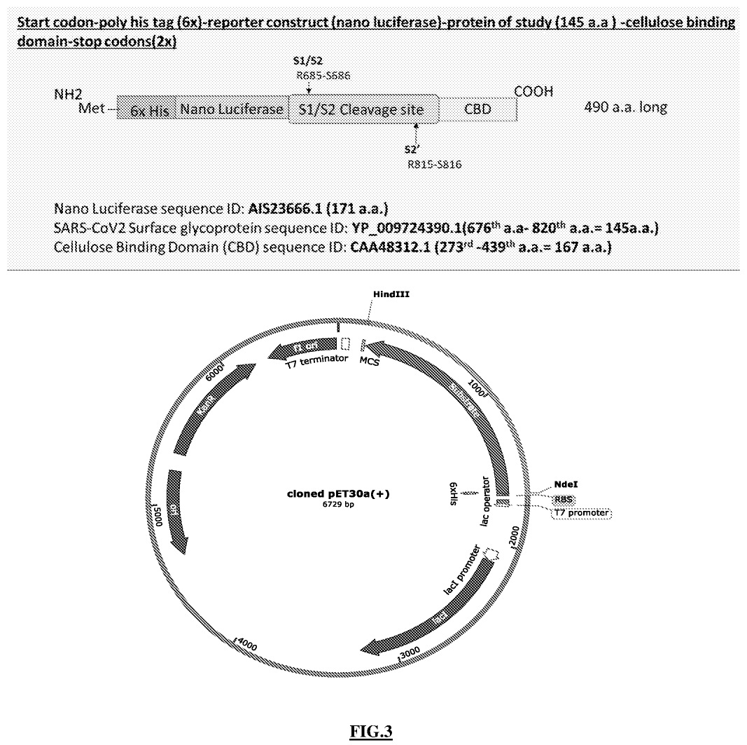

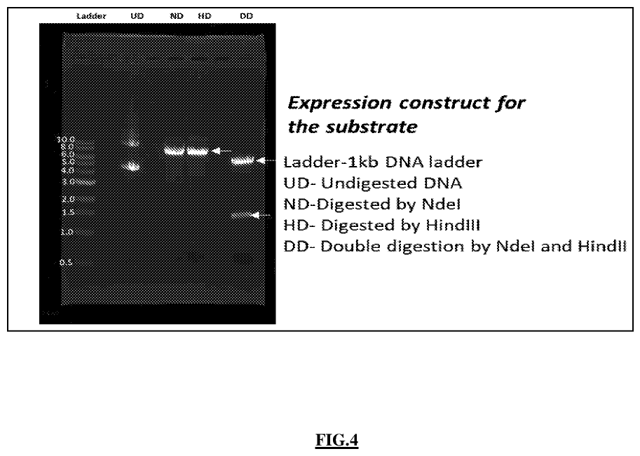

The designed gene sequence was codon optimized for expression in bacterial expression system and the entire fusion insert was cloned in bacterial expression vector pET30a between restriction enzyme sites NdeI and HindIII [FIGS. 3 and 4].

Details about cloning are as follows:
Substrate DNA (Seq. Id no 2) (URSCREENSARS-COV-2BactDNA) designed is comprised of Start codon (Methionine)-poly his tag (6x)-reporter construct (nano luciferase) (Seq. Id no 3) (URSCREENSARS-COV2NanoLucDNA)-protease cleavage site (seq. id number 4) (URSCREENSARS-COV2cleavagesiteDNA)-cellulose binding domain (Seq. Id no 5) (URSCREENSARS-COV2CBDDNA)-stop codons. [FIG. 3]
Accession number for source gene sequence used for Nano luciferase is AIS23666. [6]
Accession number for source gene sequence used for Cellulose binding domain is CAA48312.1 [7]. Accession number for source gene sequence used for protease cleavage site is YP_009724390.1 [8]. Start codon methionine was introduced in the sequence to enable start of protein translation.
Methionine was followed by introduction of hexa histidine residues meant for purification of expressed protein
Two stop codons were introduced (TAG and TAA) at the 3′ end of the cellulose binding domain sequence.
Cleavage site for restriction endonuclease NdeI was introduced at the 5′ end of the sequence before methionine start codon. Only three nucleotides (CAT) had to be added extra before ATG because restriction enzyme cleavage site is CATATG and the remaining part of the same i.e. ATG was available from the start codon itself.
Cleavage site for restriction endonuclease HindIII (AAGCTT) was introduced at the 3′ end of the sequence after stop codons.
The entire fusion gene was codon optimised for bacterial expression using IDT tool (Integrated DNA technologies) and the sequences (original and codon optimized) were aligned using using CLUSTAL OMEGA tool to check for any changes in ultimate protein expression.
The gene sequence was inserted in bacterial expression vector Pet30a+ between NdeI and HindIII restriction sites.
The cloning was further validated by restriction digestion of the plasmid DNA using NdeI and HindIII restriction enzymes

### Example 2

### Substrate Protein Expression and Purification

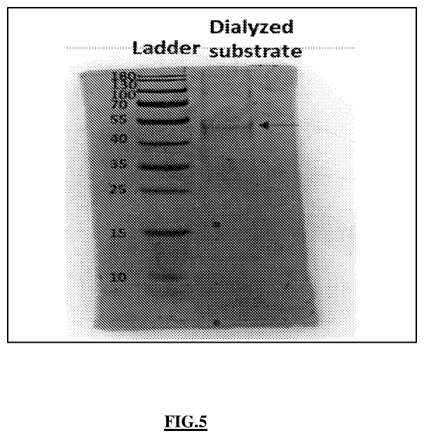

We expressed the substrate protein (Seq Id no. 1) (URSCREENSARS-COV-2) in E. coli Nico21 competent cells (Procured from New England Biolabs, 240 Country Road, Ipswich, MA 01938-2723, United States) and purified using Ni-NTA purification technique. The substrate could be purified from the soluble fraction. The substrate expressing plasmid was transformed in Nico21 cells and plated in LB-Kan plate. The plate was incubated overnight at 37 degree Celsius and one of the transformed colonies was added to 2 ml of selection media (primary inoculation). After growing for 4-6 hrs, the secondary inoculation was performed in 200 ml culture and the culture was allowed to grow till OD600 0.4-0.5 is reached. The culture was then induced by adding 0.5 mM IPTG and incubated for 16 hrs at 15 degree Celsius under shaking condition. After incubation, the cells were collected by spinning down the culture at 4 degrees. The cells were re suspended in 20 ml cell lysis buffer (50 mM NaH2PO4, 300 mM NaCl, 10 mM Imidazole pH 8) and was sonicated (30 Amp 20 sec on 20 sec off condition for 60 minutes). The soluble (cytoplasmic) and insoluble (inclusion bodies and debris) fractions were separated by centrifuging the lysate at 15000 g for 30 minutes at 4 degrees. The pellet and the supernatant were run in 10% resolving SDS-PAGE gel (4% stacking used) to check protein expression. Gel was run at 70V for 15 minutes followed by 110V till the protein ladder resolved. The gel was stained with Simply Blue gel stain (Invitrogen) and viewed in ChemiDoc. The soluble fraction obtained was used to purify the protein using Ni-NTA column (Qiagen). At first, the column was placed in QIA rack at RT to let the resin descend to the end of the column. The seal was broken before opening the cap and the storage buffer was allowed to flow through. The column was equilibrated by adding 10 ml of binding buffer (the binding buffer used was same as cell lysis buffer) and allowed it to pass through by gravity flow. The soluble fraction was added to column and let stay for 5 minutes. Then it was let pass through. The column was washed using wash buffer and protein eluted using elution buffer. Wash buffer and elution buffer contained 20 mM Imidazole 250 mM Imidazole respectively, keeping rest conditions same as cell lysis buffer. After purification, the protein was dialyzed in dialysis buffer (50 mM Tris pH7.4, 100 mM KCl, 20% glycerol, 7 mM beta mercaptoethanol) overnight at 4 degree Celsius. The dialyzed protein was run on SDS-PAGE and checked using gel staining [FIG. 5], aliquoted in multiple tubes and stored at −80 degree Celsius.

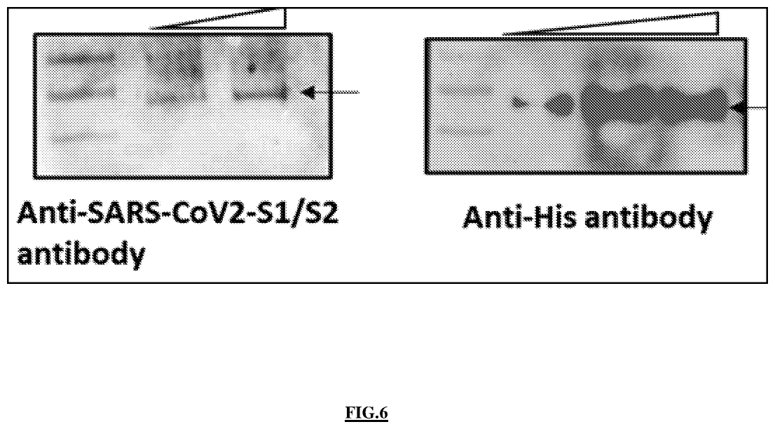

The purified substrate protein was also validated using western blot analyses using both anti-SARS-COV2 S1/S2 antibody as well as anti-His antibody [FIG. 6]. Purified protein along with SDS-gel loading dye was loaded in 10% resolving SDS-PAGE gel (4% stacking used). The gel was run at 70V for 15 minutes followed by run at 110V till the ladder resolved. The gel was transferred using nitrocellulose membrane (pore size 0.45 um) in transfer buffer (190 mM glycine, 25 mM tris pH 8.3, 200 ml methanol) at 400 mA for 2hrs at 4-degree Celsius condition. The blot was stained using Ponceau staining solution (1% Ponceau stain in 5% acetic acid) to view the status of transfer. After confirmation of protein transfer, the membrane was blocked using 5% skimmed milk in TBST buffer (20 mM Tris, 150 mM NaCl, 0.1% Tween 20 Ph7.6) for 2 hours in slow speed rocking condition at room temperature. Primary antibody (anti SARS-COV2 S2/S2′ site raised in rabbit, Gene Tex GTX1353861:1000 dilution in 5% blocking solution) (or anti-His antibody) was added to the blot and incubated at 4 degree Celsius for 14 hours in slow speed rocking condition. The blot was further incubated in primary antibody for 15 minutes at room temperature in rocking condition and then washed with TBST buffer for 5 minutes, three times in high-speed rocking condition. The blot was then incubated with secondary antibody (anti rabbit IgG raised in goat NBP175346 1:5000 in TBST buffer) for 1 hour at room temperature in slow speed rocking condition. The washing step was repeated. The blot was developed in ChemiDoc using Clarity ECL substrate.

### Example 3

### Cleavage Assay

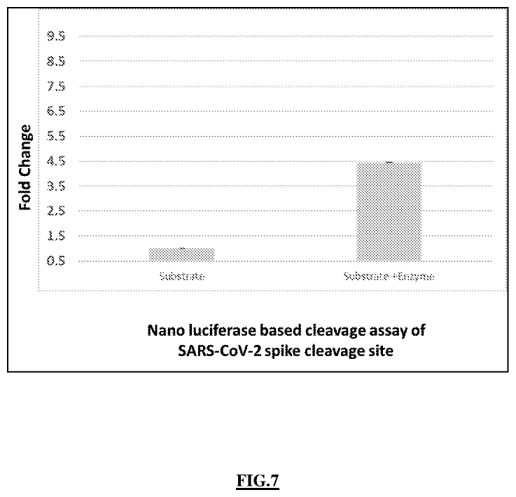

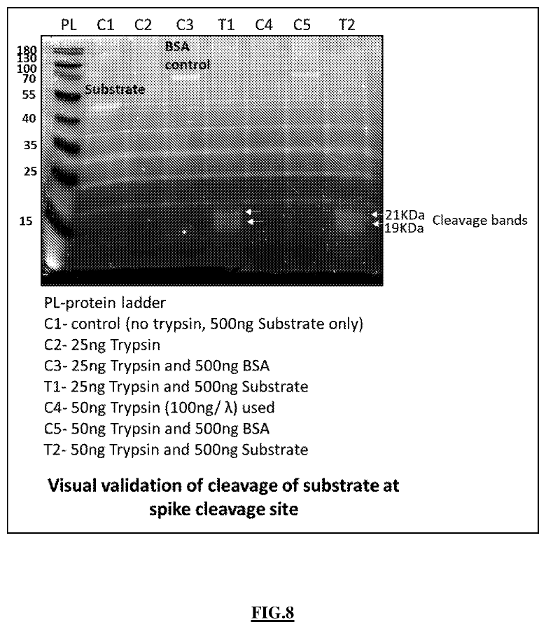

We first used trypsin to check if the substrate was getting cleaved and also whether luciferase was active. We used cellulose slurry (Sigma cell cellulose type 50 used to make slurry in buffer used for reaction at 33% w/v slurry composition) to trap the cellulose binding domain of the substrate after the trypsin treatment followed by centrifugation and recovery of the supernatant. Luminescence was recorded. More luciferase activity was observed in case of enzyme treated substrate versus substrate without enzyme treatment. The assay was performed in triplicates for five independent times to ensure the reproducibility. Thus, it was established that our substrate is active [FIG. 7]. To additionally confirm the cleavage of the substrate, we also ran cleavage assay reactions (reaction in 50 μl of 0.1M Tris pH7.45, 37 degree Celsius for 1 hour) on 15% SDS-PAGE. Bovine serum albumin (BSA) was used as a control to check if cleavage is non-specific. After gel running, the gel was stained with Sypro Ruby protein stain. We could clearly see the cleavage bands of expected molecular weight after gel staining [FIG. 8]. So, the substrate cleavage was confirmed both using visual analyses as well as luciferase assay.

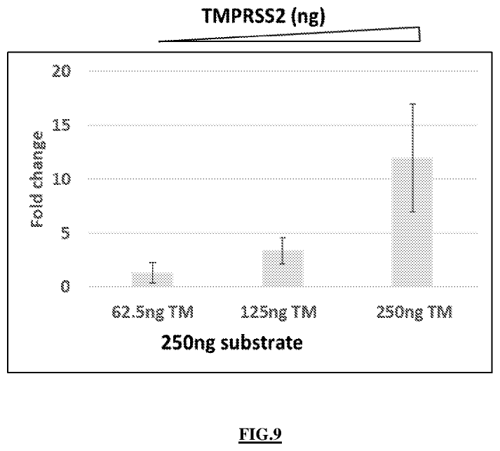

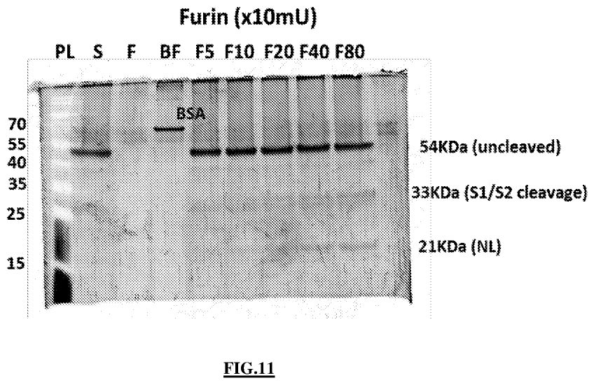

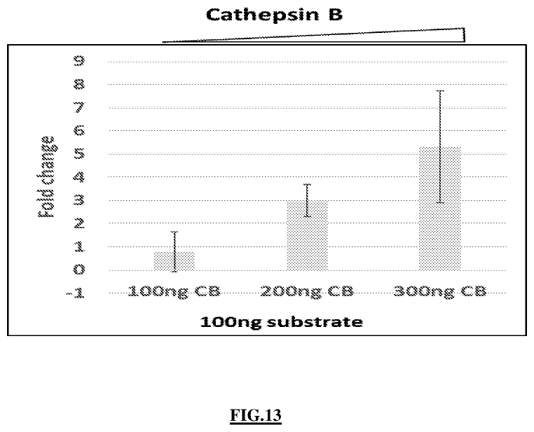

We further used few other proteases as well i.e. TMPRSS2 [FIG. 9, 10], Furin [FIG. 11, 12] and Cathepsin B [FIG. 13, 14] to validate our assay system. We have used inhibitors for these proteases as well to demonstrate that when the inhibitors are used the enzyme activities are indeed inhibited. Representative results corresponding to each of the additional enzymes tested are enlisted.

Henceforth, luciferase assay will be considered to scale up this assay system to a high-throughput form.
1. Designing the concept of mode of action of the assay system
2. Designing fusion protein construct for substrate used in the assay system
3. Codon modification of substrate encoding DNA for expression in bacteria
4. Cloning substrate gene in bacterial expression plasmid and substrate protein expression followed by purification from bacterial culture.
5. Validation of substrate cleavage by trypsin/trypsin-like protease using trypsin, TMPRSS2, Furin and Cathepsin B as representative enzymes.
6. Validation of cleavage assay both using gel based detection as well as reporter assay.

### ADVANTAGES

1. It is an in vitro assay system
2. The substrate is purified from bacterial culture and thus purification is easy.
3. Substrate cleavage assay does not require animal or human cell culture
4. Infectious virus is not involved and thus the assay system is safe.
5. The assay system is animal cell culture independent and thus less time consuming
6. The assay system can be upgraded to a high-throughput system and thus it will help in screening multiple drugs at a time.
7. It can also be used to identify if any other unknown host protease involved in spike protein cleavage. The designed assay system is easy to understand and interpret with no complicated steps involved.

### REFERENCES

1. Kleine-Weber H, Elzayat MT, Hoffmann M, Pohlmann S. Functional analysis of potential cleavage sites in the MERS-coronavirus spike protein. Sci Rep. 2018 Nov. 9;8 (1): 16597. doi: 10.1038/s41598-018-34859-w. PMID: 30413791; PMCID: PMC6226446.

2. Kam YW, Okumura Y, Kido H, Ng LF, Bruzzone R, Altmeyer R. Cleavage of the SARS coronavirus spike glycoprotein by airway proteases enhances virus entry into human bronchial epithelial cells in vitro. PLOS One. 2009 Nov. 17;4 (11): e7870. doi: 10.1371/journal.pone.0007870. PMID: 19924243; PMCID: PMC2773421.

3. Hoffmann M, Kleine-Weber H, Schroeder S, Krüger N, Herrler T, Erichsen S, Schiergens TS, Herrler G, Wu NH, Nitsche A, Müller MA, Drosten C, Pöhlmann S. SARS-COV-2 Cell Entry Depends on ACE2 and TMPRSS2 and Is Blocked by a Clinically Proven Protease Inhibitor. Cell. 2020 Apr. 16;181 (2): 271-280.e8. doi: 10.1016/j.cell.2020.02.052. Epub 2020 Mar. 5. PMID: 32142651; PMCID: PMC7102627.

4. Hoffmann M, Kleine-Weber H, Pohlmann S. A Multibasic Cleavage Site in the Spike Protein of SARS-COV-2 Is Essential for Infection of Human Lung Cells. Mol Cell. 2020 May 21;78 (4): 779-784.e5. doi: 10.1016/j.molcel.2020.04.022. Epub 2020 May 1. PMID: 32362314; PMCID: PMC7194065.

5. Jaimes JA, Millet JK, Whittaker GR. Proteolytic Cleavage of the SARS-COV-2 Spike Protein and the Role of the Novel S1/S2 Site. iScience. 2020 June 26;23 (6): 101212. doi: 10.1016/j.isci.2020.101212. Epub 2020 May 28. PMID: 32512386; PMCID: PMC7255728.

6. Hall, M.P. (2012). Engineered Luciferase Reporter from a Deep Sea Shrimp Utilizing a Novel Imidazopyrazinone Substrate. ACS Chem. Biol. 7 (11), 1848-1857, doi: 0.1021/cb3002478

7. Morag, E (1995). Expression, Purification, and Characterization of the CelluloseBinding Domain of the Scaffoldin Subunit from the Cellulosome of Clostridium thermocellum. Applied and environmental microbiology, 61 (5),1980-1986

8. Wu F, Zhao S, Yu B, Chen YM, Wang W, Song ZG, Hu Y, Tao ZW, Tian JH, Pei YY, Yuan ML, Zhang YL, Dai FH, Liu Y, Wang QM, Zheng JJ, Xu L, Holmes EC, Zhang YZ. A new coronavirus associated with human respiratory disease in China. Nature. 2020 March;579 (7798): 265-269. doi: 10.1038/s41586-020-2008-3. Epub 2020 Feb. 3. Erratum in: Nature. 2020 April;580 (7803): E7. PMID: 32015508; PMCID: PMC7094943.

1-10. (canceled)

11. A recombinant construct for screening drugs against SARS-COV-2 spike protein having SEQ ID NO:2.

12. The recombinant construct of claim 11, comprising:
(i) a fusion protein having SEQ ID NO:1
(ii) a designed substrate; and
(iii) a reporter protein.

13. The recombinant construct of claim 12, wherein the designed substrate carries a hexa histidine tag at one end for protein purification.

14. The recombinant construct of claim 12, wherein methionine is added at the N terminus of hexa histidine.

15. The recombinant construct of claim 12, wherein the reporter protein comprises:
(a) a start codon methionine at the N terminus of hexa histidine residues; and
(b) two stop codons at the 3′ end of a cellulose binding domain sequence.

16. The recombinant construct of claim 12, wherein the fusion protein comprises:
(a) a reporter protein having SEQ ID NO:3;
(b) a spike protein cleavage sequence having SEQ ID NO:4; and
(c) a cellulose binding domain having SEQ ID NO:5.

17. A method for drug screening against SARS-COV-2 with an in vitro assay system, the method comprising:
(i) providing a fusion protein having SEQ ID NO:1;
(ii) adding trypsin like protease and a drug to be screened to the fusion protein of (i) to obtain a reaction mixture;
(iii) providing a cellulose matrix/slurry;
(iv) mixing the reaction mixture of (ii) with the cellulose matrix/slurry of (iii) to obtain a second mixture;
(v) centrifuging the second mixture of (iv) to obtain a reporter protein from a reaction supernatant for a reporter assay; and
(vi) measuring a reporter signal, wherein decrease in reporter signal as compared with no inhibitor control confirms positive activity of the drug to be screened.

18. The method of claim 17, wherein the drug is selected from the group consisting of Camostat mesylate, cathepsin B inhibitor, furin inhibitor II, protease inhibitors, Furin inhibitor I, trypsin inhibitors, Ovomucoid, Kunitz Trypsin Inhibitor, serine protease inhibitors, and TMPRSS2 inhibitors.

19. The method of claim 17, wherein the cellulose matrix/slurry comprises cellulose in a buffer used for reaction as a 33% w/v slurry composition.

20. The method of claim 17, wherein the reporter protein is selected from the group comprising of luciferase and fluorescent reporter.
